(19) United States
(12) Patent Application Publication
(10) Pub. No.: US 2026/0201461 A1
(43) Pub. Date: Jul. 16, 2026

(54) SYSTEMS AND METHODS FOR EVALUATION OF EXPRESSION PATTERNS

(71) Applicant: Orion Medicines, Inc., San Francisco, CA (US)

(72) Inventor: Francois Aguet, San Diego, CA (US)

(73) Assignee: Orion Medicines, Inc., San Francisco, CA (US)

(21) Appl. No.: 19/139,310

(22) PCT Filed: Dec. 16, 2023
(86) PCT No.: PCT/US2023/084479

§ 371 Date: Jun. 13, 2025

Related U.S. Application Data
(60) Provisional application No. 63/433,422, filed on Dec. 16, 2022.

(51) Int. Cl. C12Q 1/6869; C12N 15/113; C12N 15/63; C12Q 1/6883; C40B 70/00; G16B 20/20; G16B 30/00; G16B 40/20

(52) U.S. Cl. CPC C12Q 1/6869; C12N 15/113; C12N 15/63; C12Q 1/6883; C40B 70/00; G16B 20/20; G16B 30/00; G16B 40/20; C12N 2310/14; C12N 2310/20; C12Q 2600/156

(57) ABSTRACT

Presented herein are methods for mapping the effects of gene variants using high throughput sequencing and machine learning to determine the pathogenicity of each variant

### CROSS-REFERENCE TO RELATED PATENT APPLICATION

This application claims the benefit under 35 U.S.C. § 119(e) of U.S. Provisional Application No. 63/433,422, filed Dec. 16, 2022, the entire contents of which are incorporated herein by reference.

### FIELD OF TECHNOLOGY

The technology disclosed relates to artificial intelligence type computers and digital data processing systems and corresponding data processing methods and products for emulation of intelligence (i.e., knowledge-based systems, reasoning systems, and knowledge acquisition systems); and including systems for reasoning with uncertainty (e.g., fuzzy logic systems), adaptive systems, machine learning systems, and artificial neural networks. In particular, the technology disclosed relates to using techniques for converting context of an artificial neural network (ANN) or another type of computing system that is trainable through machine learning.

Additionally, the technology disclosed relates to pre-processing of inputs for artificial intelligence type computers and digital data processing systems and corresponding data processing methods and products for emulation of intelligence as well as the actual pre-processed inputs themselves.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Genomics, in the broad sense, also referred to as functional genomics, aims to characterize the function of every genomic element of an organism by using genome-scale assays such as genome sequencing, transcriptome profiling and proteomics. Genomics arose as a data-driven science-it operates by discovering novel properties from explorations of genome-scale data rather than by testing preconceived models and hypotheses. Applications of genomics include finding associations between genotype and phenotype, discovering biomarkers for patient stratification, predicting the function of genes, and charting biochemically active genomic regions such as transcriptional enhancers.

Genomics data are too large and too complex to be mined solely by visual investigation of pairwise correlations. Instead, analytical tools are required to support the discovery of unanticipated relationships, to derive novel hypotheses and models and to make predictions. Unlike some algorithms, in which assumptions and domain expertise are hard coded, machine learning algorithms are designed to automatically detect patterns in data. Hence, machine learning algorithms are suited to data-driven sciences and, in particular, to genomics. However, the performance of machine learning algorithms can strongly depend on how the data are represented, that is, on how each variable (also called a feature) is computed. For instance, to classify a tumor as malign or benign from a fluorescent microscopy image, a preprocessing algorithm could detect cells, identify the cell type, and generate a list of cell counts for each cell type.

A machine learning model can take the estimated cell counts, which are examples of handcrafted features, as input features to classify the tumor. A central issue is that classification performance depends heavily on the quality and the relevance of these features. For example, relevant visual features such as cell morphology, distances between cells or localization within an organ are not captured in cell counts, and this incomplete representation of the data may reduce classification accuracy.

Deep learning, a subdiscipline of machine learning, addresses this issue by embedding the computation of features into the machine learning model itself to yield end-to-end models. This outcome has been realized through the development of deep neural networks, machine learning models that comprise successive elementary operations, which compute increasingly more complex features by taking the results of preceding operations as input. Deep neural networks are able to improve prediction accuracy by discovering relevant features of high complexity, such as the cell morphology and spatial organization of cells in the above example. The construction and training of deep neural networks have been enabled by the explosion of data, algorithmic advances, and substantial increases in computational capacity, particularly through the use of graphical processing units (GPUs).

The goal of supervised learning is to obtain a model that takes features as input and returns a prediction for a so-called target variable. An example of a supervised learning problem is one that predicts whether an intron is spliced out or not (the target) given features on the RNA such as the presence or absence of the canonical splice site sequence, the location of the splicing branchpoint or intron length. Training a machine learning model refers to learning its parameters, which commonly involves minimizing a loss function on training data with the aim of making accurate predictions on unseen data.

For many supervised learning problems in computational biology, the input data can be represented as a table with multiple columns, or features, each of which contains numerical or categorical data that are potentially useful for making predictions. Some input data are naturally represented as features in a table (such as temperature or time), whereas other input data need to be first transformed (such as deoxyribonucleic acid (DNA) sequence into k-mer counts) using a process called feature extraction to fit a tabular representation. For the intron-splicing prediction problem, the presence or absence of the canonical splice site sequence, the location of the splicing branchpoint and the intron length can be preprocessed features collected in a tabular format. Tabular data are standard for a wide range of supervised machine learning models, ranging from simple linear models, such as logistic regression, to more flexible nonlinear models, such as neural networks and many others.

Logistic regression is a binary classifier, that is, a supervised learning model that predicts a binary target variable. Specifically, logistic regression predicts the probability of the positive class by computing a weighted sum of the input features mapped to the [0,1] interval using the sigmoid function, a type of activation function. The parameters of logistic regression, or other linear classifiers that use different activation functions are the weights in the weighted sum. Linear classifiers fail when the classes, for instance, that of an intron spliced out or not, cannot be well discriminated with a weighted sum of input features. To improve predictive performance, new input features can be manually added by transforming or combining existing features in new ways, for example, by taking powers or pairwise products.

Neural networks use hidden layers to learn these nonlinear feature transformations automatically. Each hidden layer can be thought of as multiple linear models with their output transformed by a nonlinear activation function, such as the sigmoid function or the more popular rectified-linear unit (ReLU). Together, these layers compose the input features into relevant complex patterns, which facilitates the task of distinguishing two classes.

Deep neural networks use many hidden layers, and a layer is said to be fully-connected when each neuron receives inputs from all neurons of the preceding layer. Neural networks are commonly trained using stochastic gradient descent, an algorithm suited to training models on very large data sets. Implementation of neural networks using modern deep learning frameworks enables rapid prototyping with different architectures and data sets. Fully-connected neural networks can be used for a number of genomics applications, which include predicting the percentage of exons spliced in for a given sequence from sequence features such as the presence of binding motifs of splice factors or sequence conservation; prioritizing potential disease-causing genetic variants; and predicting cis-regulatory elements in a given genomic region using features such as chromatin marks, gene expression and evolutionary conservation.

Local dependencies in spatial and longitudinal data must be considered for effective predictions. For example, shuffling a DNA sequence or the pixels of an image severely disrupts informative patterns. These local dependencies set spatial or longitudinal data apart from tabular data, for which the ordering of the features is arbitrary. Consider the problem of classifying genomic regions as bound versus unbound by a particular transcription factor, in which bound regions are defined as high-confidence binding events in chromatin immunoprecipitation following by sequencing (ChIP-seq) data. Transcription factors bind to DNA by recognizing sequence motifs. A fully-connected layer based on sequence-derived features, such as the number of k-mer instances or the position weight matrix (PWM) matches in the sequence, can be used for this task. As k-mer or PWM instance frequencies are robust to shifting motifs within the sequence, such models could generalize well to sequences with the same motifs located at different positions. However, they would fail to recognize patterns in which transcription factor binding depends on a combination of multiple motifs with well-defined spacing. Furthermore, the number of possible k-mers increases exponentially with k-mer length, which poses both storage and overfitting challenges.

A convolutional layer is a special form of fully-connected layer in which the same fully-connected layer is applied locally, for example, in a 6 bp window, to all sequence positions. This approach can also be viewed as scanning the sequence using multiple PWMs, for example, for transcription factors GATA1 and TAL1. By using the same model parameters across positions, the total number of parameters is drastically reduced, and the network is able to detect a motif at positions not seen during training. Each convolutional layer scans the sequence with several filters by producing a scalar value at every position, which quantifies the match between the filter and the sequence. As in fully-connected neural networks, a nonlinear activation function (commonly ReLU) is applied at each layer. Next, a pooling operation is applied, which aggregates the activations in contiguous bins across the positional axis, commonly taking the maximal or average activation for each channel. Pooling reduces the effective sequence length and coarsens the signal. The subsequent convolutional layer composes the output of the previous layer and is able to detect whether a GATA1 motif and TAL1 motif were present at some distance range. Finally, the output of the convolutional layers can be used as input to a fully-connected neural network to perform the final prediction task. Hence, different types of neural network layers (e.g., fully-connected layers and convolutional layers) can be combined within a single neural network.

Convolutional neural networks (CNNs) can predict various molecular phenotypes on the basis of DNA sequence alone. Applications include classifying transcription factor binding sites and predicting molecular phenotypes such as chromatin features, DNA contact maps, DNA methylation, gene expression, translation efficiency, RBP binding, and microRNA (miRNA) targets. In addition to predicting molecular phenotypes from the sequence, convolutional neural networks can be applied to more technical tasks traditionally addressed by handcrafted bioinformatics pipelines. For example, convolutional neural networks can predict the specificity of guide RNA, denoise ChIP-seq, enhance Hi-C data resolution, predict the laboratory of origin from DNA sequences and call genetic variants. Convolutional neural networks have also been employed to model long-range dependencies in the genome. Although interacting regulatory elements may be distantly located on the unfolded linear DNA sequence, these elements are often proximal in the actual 3D chromatin conformation. Hence, modelling molecular phenotypes from the linear DNA sequence, albeit a crude approximation of the chromatin, can be improved by allowing for long-range dependencies and allowing the model to implicitly learn aspects of the 3D organization, such as promoter-enhancer looping. This is achieved by using dilated convolutions, which have a receptive field of up to 32 kb. Dilated convolutions also allow splice sites to be predicted from sequence using a receptive field of 10 kb, thereby enabling the integration of genetic sequence across distances as long as typical human introns (See Jaganathan, K. et al. Predicting splicing from primary sequence with deep learning. Cell 176, 535-548 (2019)).

Different types of neural network can be characterized by their parameter-sharing schemes. For example, fully-connected layers have no parameter sharing, whereas convolutional layers impose translational invariance by applying the same filters at every position of their input. Recurrent neural networks (RNNs) are an alternative to convolutional neural networks for processing sequential data, such as DNA sequences or time series, that implement a different parameter-sharing scheme. Recurrent neural networks apply the same operation to each sequence element. The operation takes as input the memory of the previous sequence element and the new input. It updates the memory and optionally emits an output, which is either passed on to subsequent layers or is directly used as model predictions. By applying the same model at each sequence element, recurrent neural networks are invariant to the position index in the processed sequence. For example, a recurrent neural network can detect an open reading frame in a DNA sequence regardless of the position in the sequence. This task requires the recognition of a certain series of inputs, such as the start codon followed by an in-frame stop codon.

The main advantage of recurrent neural networks over convolutional neural networks is that they are, in theory, able to carry over information through infinitely long sequences via memory. Furthermore, recurrent neural networks can naturally process sequences of widely varying length, such as mRNA sequences. However, convolutional neural networks combined with various tricks (such as dilated convolutions) can reach comparable or even better performances than recurrent neural networks on sequence-modelling tasks, such as audio synthesis and machine translation. Recurrent neural networks can aggregate the outputs of convolutional neural networks for predicting single-cell DNA methylation states, RBP binding, transcription factor binding, and DNA accessibility. Moreover, because recurrent neural networks apply a sequential operation, they cannot be easily parallelized and are hence much slower to compute than convolutional neural networks.

Each human has a unique genetic code, though a large portion of the human genetic code is common for all humans. In some cases, a human genetic code may include an outlier, called a genetic variant, that may be common among individuals of a relatively small group of the human population. For example, a particular human protein may comprise a specific sequence of amino acids, whereas a variant of that protein may differ by one amino acid in the otherwise same specific sequence.

Genetic variants may be pathogenetic, leading to diseases. Though most of such genetic variants have been depleted from genomes by natural selection, an ability to identify which genetic variants are likely to be pathogenic can help researchers focus on these genetic variants to gain an understanding of the corresponding diseases and their diagnostics, treatments, or cures. The clinical interpretation of millions of human genetic variants remains unclear. Some of the most frequent pathogenic variants are single nucleotide missense mutations that change the amino acid of a protein. However, not all missense mutations are pathogenic.

Models that can predict molecular phenotypes directly from biological sequences can be used as in silico perturbation tools to probe the associations between genetic variation and phenotypic variation and have emerged as new methods for quantitative trait loci identification and variant prioritization. These approaches are of major importance given that the majority of variants identified by genome-wide association studies of complex phenotypes are non-coding, which makes it challenging to estimate their effects and contribution to phenotypes. Moreover, linkage disequilibrium results in blocks of variants being co-inherited, which creates difficulties in pinpointing individual causal variants. Thus, sequence-based deep learning models that can be used as interrogation tools for assessing the impact of such variants offer a promising approach to find potential drivers of complex phenotypes. One example includes predicting the effect of non-coding single-nucleotide variants and short insertions or deletions (indels) indirectly from the difference between two variants in terms of transcription factor binding, chromatin accessibility or gene expression predictions. Another example includes predicting novel splice site creation from sequence or quantitative effects of genetic variants on splicing.

The recent success of deep learning algorithms provide an opportunity to develop tools for automatically extracting relationships between thousands of genes. Therefore, an opportunity arises to predict variant pathogenicity of millions of variants along the entire length of a gene as input to deep neural networks.

### SUMMARY OF THE DISCLOSURE

The present disclosure encompasses the discovery that the effects of variants can be mapped using high throughput sequencing and machine learning to determine the pathogenicity of each variant.

The details of one or more embodiments are set forth in the claims and the description below. Other features, objects, and advantages will be apparent from the description, and from the claims.

An aspect of the disclosure is directed to a method of simultaneously determining the effects of a plurality of variants in a gene comprising, or alternatively consisting essentially of, or yet further consisting of: a) generating a mutant expression vector for each of the plurality of variants; b) expressing each variant in a cell in a plurality of single cells; and c) sequencing the RNA expressed in each single cell and evaluating expression patterns that result in each single cell to generate single cell RNA sequencing data.

In some embodiments, the method further comprises, or alternatively consists essentially of, or yet further consists of evaluating the single cell RNA sequencing data to determine the pathogenicity of each variant.

In some embodiments, evaluating the single cell RNA sequencing data comprises, or alternatively consists essentially of, or yet further consists of use of machine learning.

In some embodiments, variants are classified as either pathogenic, likely pathogenic, likely benign, or benign.

In some embodiments, each cell of the plurality of single cells is configured to express a single variant of the plurality of variants.

In some embodiments, each single variant of the plurality of variants is expressed in more than one cell, thereby creating redundancy of the single cell RNA sequencing data.

In some embodiments, the plurality of single cells comprises, or alternatively consists essentially of, or yet further consists of at least 30,000, at least 40,000, at least 50,000, at least 60,000, at least 70,000, at least 80,000, at least 90,000 or at least 100,000 single cells.

In some embodiments, the plurality of variants comprises, or alternatively consists essentially of, or yet further consists of at least 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or at least 3000 variants.

In some embodiments, the gene is selected from genes associated with rare disease.

In some embodiments, the gene is selected from genes associated with oncology.

In some embodiments, the mutant expression vector is generated using CRISPR.

In some embodiments, the mutant expression vector is generated using CRISPRi.

In some embodiments, the mutant expression vector is generated using RNAi.

In some embodiments, the RNA from each single cell is identified using barcoding and sequencing.

In some embodiments, the method further comprises, or alternatively consists essentially of, or yet further consists of repeating steps a)-c) in more than one set of primary cells or cell lines.

Another aspect of the disclosure is directed to a method of simultaneously determining the effects of a gene comprising, or alternatively consisting essentially of, or yet further consisting of: a) perturbing the function of a gene in a cell in a plurality of single cells; and b) sequencing the RNA expressed in each single cell and evaluating expression patterns that result in each single cell to generate single cell RNA sequencing data.

In some embodiments, the method further comprises, or alternatively consists essentially of, or yet further consists of evaluating the single cell RNA sequencing data to determine the pathogenicity of each perturbed gene.

In some embodiments, perturbing the function of a gene comprises, or alternatively consists essentially of, or yet further consists of using one or more drug compounds. In some embodiments, a different drug compound is administered to each single cell.

In some embodiments, the plurality of drug compounds comprises, or alternatively consists essentially of, or yet further consists of at least 100, at least 200, at least 300, at least 500, at least 1,000, at least 2,000, at least 3,000 at least 4,000, at least 5,000, or at least 10,000 compounds.

In some embodiments, evaluating the single cell RNA sequencing data comprises, or alternatively consists essentially of, or yet further consists of use of machine learning.

Another aspect of the disclosure is directed to a method comprising, or alternatively consisting essentially of, or yet further consisting of: a) receiving RNA-seq data for a plurality of cells, each cell expressing a variant generated using a mutant expression vector; and b) using the RNA-seq data to train a machine learning model to receive, as input, RNA-seq data and provide, as output, a pathogenicity classification.

In some embodiments, the method comprises, or alternatively consists essentially of, or yet further consists of a) receiving RNA-seq data for a cell; and b) providing the RNA-seq data to a machine learning model to generate a pathogenicity classification for a variant, the machine learning model trained according to the present disclosure.

### BRIEF DESCRIPTION OF THE FIGURES

FIG. 1 is a diagram showing the workflow for using Perturb-seq to map the effects of variants with high throughput sequencing. In the first panel, constructs are generated to mutate all possible variants in a gene, and each cell gets one variant. CRISPR and Twist are used to generate the variants in a single-cell dependent fashion. In the middle panel, single cell RNA-seq is used to cluster variants with similar function, and are compared to known benign and known pathogenic variants. In the final panel, machine learning is used to determine the pathogenicity of each variant, for example, classifying variants as pathogenic, likely pathogenic, likely benign, and benign. In the example shown in the bottom panel, a function score is assigned to each variant, ranging from −4 to positive values.

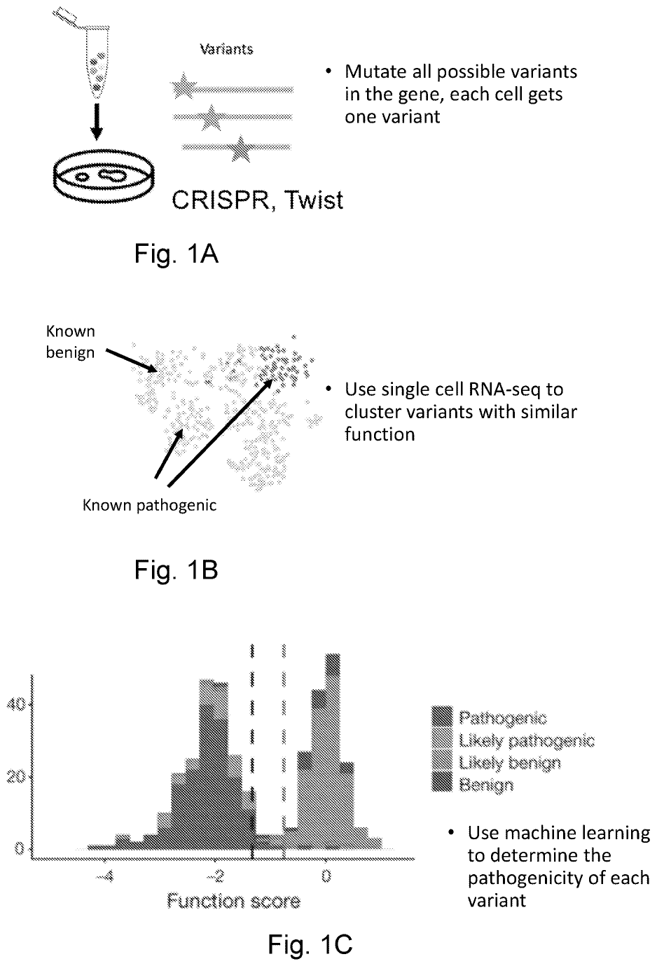

FIG. 2 is graph showing results from an example Perturb-seq workflow to map the effects of 2300 variants in the TP53 gene. The 2300 variants were expressed in 50,000 single cells, resulting in over 10-fold redundancy for each variant. The graph shows the density of variants (y-axis) over a range of predicted missense probability scores (x-axis). The range of predicted missense probability spans scores lower than 0.4 to over 0.8. Variants with a Primate AI 3D scores greater than 0.8 were classified as pathogenic variants. Another set of variants were classified as synonymous. The data show a correlation with orthogonal datasets of 0.75, which represents the state-of-the-art. Further experiments are performed on a set of prioritized genes from rare disease and oncology. The further experiments are performed using perturbation of 20,000 genes are performed using CRISPRi and 4,000 drug compounds. The further experiments are performed using ultra-high throughput barcoding and sequencing, and the biological mechanism is read out using single-cell sequencing, with an aim to characterize the effects in 100-200 primary cells and cell lines.

FIG. 3 is schematic showing the target discovery landscape. A large number of proprietary cohorts ranging from over 10,000 to over 10,000,000 are utilized to identify targets for Rare Disease, Common Disease with whole genome sequencing (WGS) combined with multiome data, and common disease with exome data only.

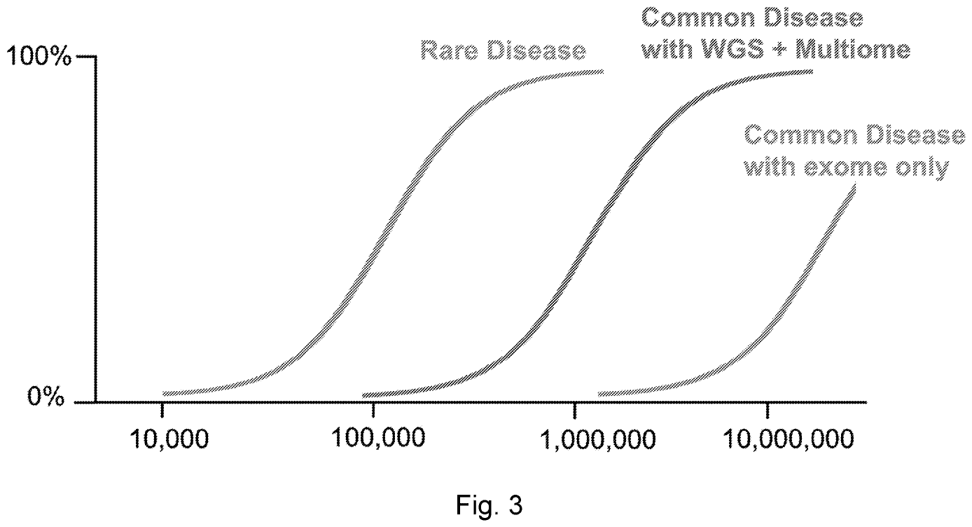

FIG. 4 is a block diagram of a computing environment for realizing the systems and methods according to embodiments of the subject matter disclosed herein.

FIG. 5 is a method flow chart illustrating an exemplary computer-based method for establishing a trained machine learning model and updating the trained model according to embodiments of the subject matter disclosed herein.

FIG. 6 is a method flow chart illustrating an exemplary computer-based method for utilizing a trained machine learning model according to embodiments of the subject matter disclosed herein.

### DETAILED DESCRIPTION

### Definitions

As it would be understood, the section or subsection headings as used herein is for organizational purposes only and are not to be construed as limiting and/or separating the subject matter described.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Techique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press).

As used in the specification and claims, the singular form “a,” “an” and “the” include plural references unless the context clearly dictates otherwise. For example, the term “a cell” includes a plurality of cells, including mixtures thereof.

“Comprising” or “comprises” is intended to mean that the compositions, for example media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this technology.

All numerical designations, e.g., pH, temperature, time, concentration, and molecular weight, including ranges, are approximations which are varied (+) or (−) by increments of 1, 5, or 10%. It is to be understood, although not always explicitly stated that all numerical designations are preceded by the term “about.” It also is to be understood, although not always explicitly stated, that the reagents described herein are merely exemplary and that equivalents of such are known in the art.

The term “about,” as used herein when referring to a measurable value such as an amount or concentration and the like, is meant to encompass variations of 20%, 10%, 5%, 1%, 0.5%, or even 0.1 % of the specified amount.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

As will be understood by one skilled in the art, for any and all purposes, all ranges disclosed herein also encompass any and all possible subranges and combinations of subranges thereof. Furthermore, as will be understood by one skilled in the art, a range includes each individual member.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

CRISPR-Cas systems, including CRISPR-Cas9 systems, as used herein, refer to non-naturally occurring systems derived from bacterial Clustered Regularly Interspaced Short Palindromic Repeats loci. These systems generally comprise an enzyme (Cas protein, such as Cas9 protein) and one or more RNAs. Said RNA is a CRISPR RNA and may be an sgRNA. Said RNA and/or said enzyme may be engineered, for example for optimal use in mammalian cells, for optimal delivery therein, for optimal activity therein, for specific uses in gene editing, etc.

As used herein, “sgRNA” refers to a CRISPR single-guide RNA. This RNA is a component of a CRISPR-Cas system. The sequence of the sgRNA determines the target sequence for gene editing, knock-down, knock-out, insertion, etc. For genome-wide approaches, it is possible to design and construct suitable sgRNA libraries. Such sgRNAs may be delivered to cells using vector delivery such as viral vector delivery.

A single CRISPR-Cas-mediated perturbation may be obtained by delivering a signle sgRNA within a single cell. In the case of sgRNA viral vector delivery, single perturbation may be obtained by delivering a single sgRNA to the cell. Number of perturbations in a cell corresponds to the MOI (multiplicity of infection). For single perturbations using CRISPR-Cas systems, one may generally implement MOI values of up to 0.5, 0.6, 0.7, or 0.8 to ensure that a cell gets only one sgRNA.

Combination of CRISPR-Cas-mediated perturbations may be obtained by delivering multiple sgRNAs within a single cell. This may be achieved in pooled format. In the case of sgRNA viral vector delivery, combined perturbation may be obtained by delivering several sgRNA vectors to the same cell. This may also be achieved in pooled format, and number of combined perturbations in a cell then corresponds to the MOI (multiplicity of infection). For combined perturbations, CRISPR-Cas systems, one may generally implement MOI values of up to 10, 12 or 15.

CRISPR interference (CRISPRi): As used herein, the term “CRISPRi” refers to a genetic perturbation technique that allows for sequence-specific repression or activation of gene expression in prokaryotic and eukaryotic cells using CRISPR complexes. CRISPRi regulates gene expression primarily on the transcriptional level.

### Modes for Carrying out the Disclosure

The present disclosure provides improved techniques for detecting and characterizing the pathogenicity of any of a variety of variants.

As described in Adamson, et al. (2016). “A Multiplexed Single-Cell CRISPR Screening Platform Enables Systematic Dissection of the Unfolded Protein Response” (Cell. 167 (7): 1867-1882.e21) and Dixit, et al. (2016). “Perturb-Seq: Dissecting Molecular Circuits with Scalable Single-Cell RNA Profiling of Pooled Genetic Screens” (Cell. 167 (7): 1853-1866.e17), the content of each is hereby incorporated by reference in its entirety, a high-throughput methodology can be used to identify the functions and relationships of a number of genes. The embodiments presented herein expand upon those initial discoveries, and show that the effects of variants can be mapped using high throughput sequencing and machine learning to determine the pathogenicity of each variant.

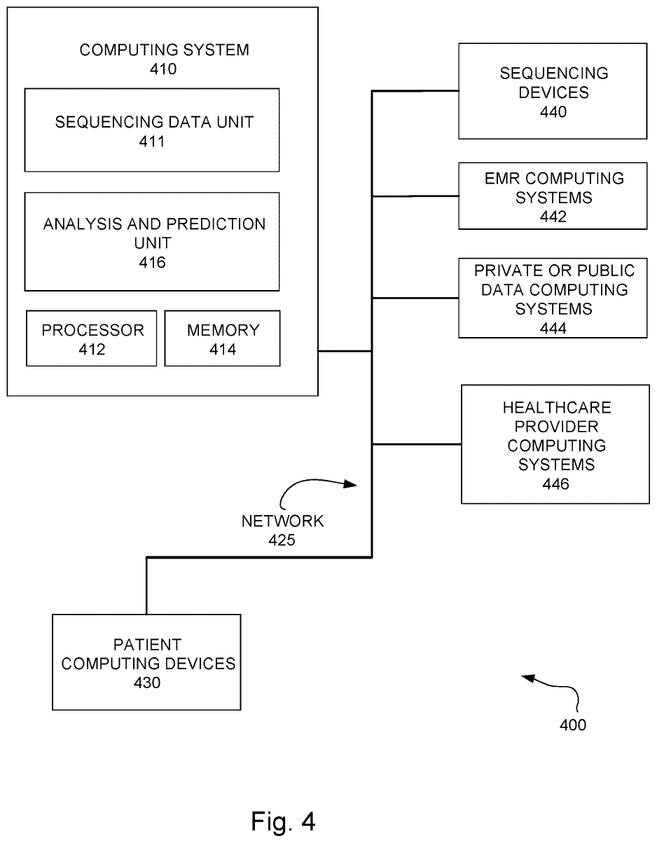

Turning attention to FIG. 4 is a block diagram of a computing environment for realizing the systems and methods for evaluating single-cell RNA-seq data to determine pathogenicity of each variant according to embodiments of the subject matter disclosed herein. RNA-seq data can include, for example, RNA sequences for RNA transcripts in an RNA profile, and/or relative abundance of RNA transcripts, indicative of gene expression. The RNA-seq data can be indicative of RNA expression patterns resulting from a gene variant, for example. The methods described herein allow for high-throughput discovery of pathogenicity of previously-unknown variants of genes. Also, the disclosed approach provides a more efficient, effective, and lower-cost way of characterizing variants, in contrast to prior methods, which required testing and analyzing variants on an individual basis.

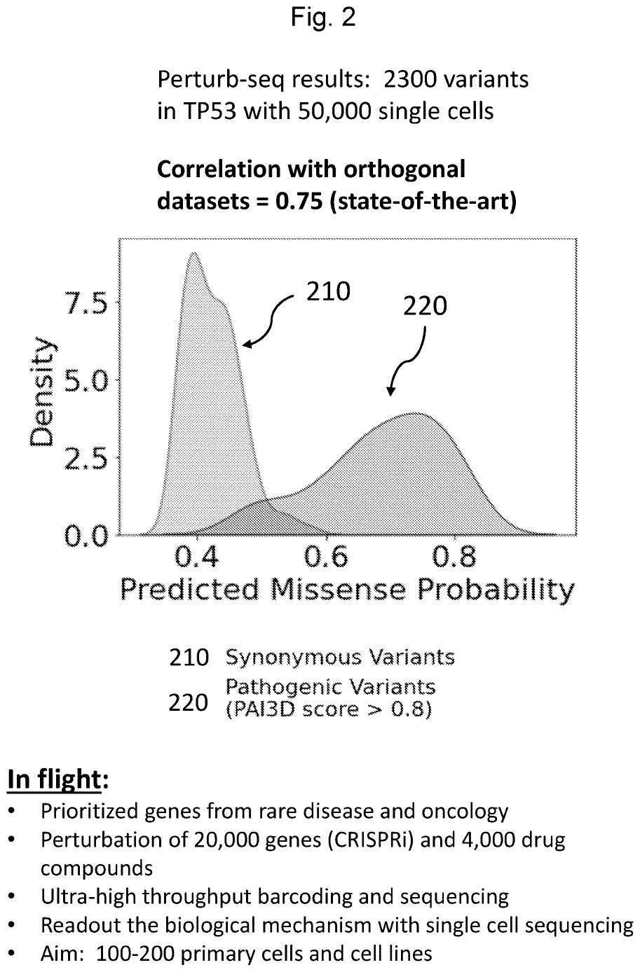

A trained machine learning model as described herein may also be useful for detecting patients who are or will be, for example, (i) responsive or nonresponsive to a particular treatment or therapy for a particular disease, and/or (ii) at higher or lower risk of a particular disease. The present technology greatly improves high-throughput structure-function analysis of gene variants. The disclosed approach enables accurate high-throughput analyses of gene variants to a degree that could not have been previously achieved. The present approach does not necessarily require a priori knowledge of a particular gene variant and enables a more unbiased analysis. The disclosed machine learning embodiments enable detection of expression patterns from vast amounts of sequencing data. For instance, in FIG. 2, the machine learning model efficiently analyzes 2300 unique variants of TP53 using single-cell RNA-seq (transcriptome) data from 50,000 cells to predict pathogenicity of each unique variant of TP53.

The overall computing environment 400 may be generally comprised of several sets of computing devices that are all communicatively coupled to each other through a computing network 425, such as the Internet, though the network 425 may be a local Intranet or a virtual private network or the like. These generalized categories of the coupled computing devices and/or systems include an RNA data analysis computing system 410, one or more patient computing-devices 430, and one or more data-service computing devices, such as public or public data collection systems 444, Electronic Medical Record (EMR)/Electronic Health Record (EHR) systems 442, and healthcare provider computing systems 446. Other data-collection and/or data provision services (such as government information service computing systems, research institution computing systems, and the like) are contemplated but not shown in this figure here for brevity. Collectively, these computing devices may be used to receive and send patient data, sequencing data, diagnostic data, and/or therapeutics data. The computing system 410, includes one or more local processors 412 that utilizes one or more memories 414 in conjunction with a sequencing data unit 411 and an analysis and prediction unit 416.

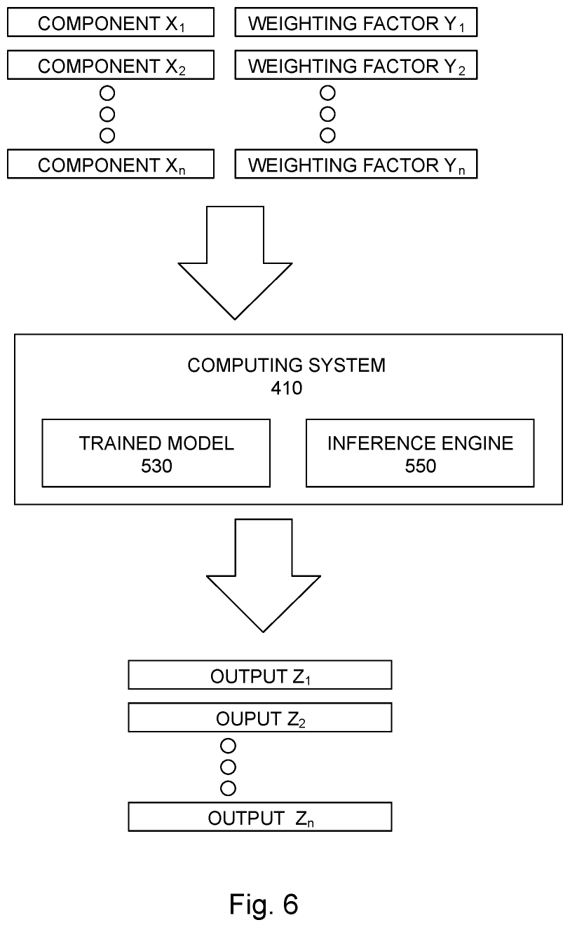

Utilizing the overall system 400 of FIG. 4, one may take advantage of sequencing and other data to inform analysis and prediction engine 416 leading to greater accuracy and predictability for further testing, diagnostics, and therapeutics. This may be accomplished by training a prediction engine as discussed further below with respect to FIG. 6.

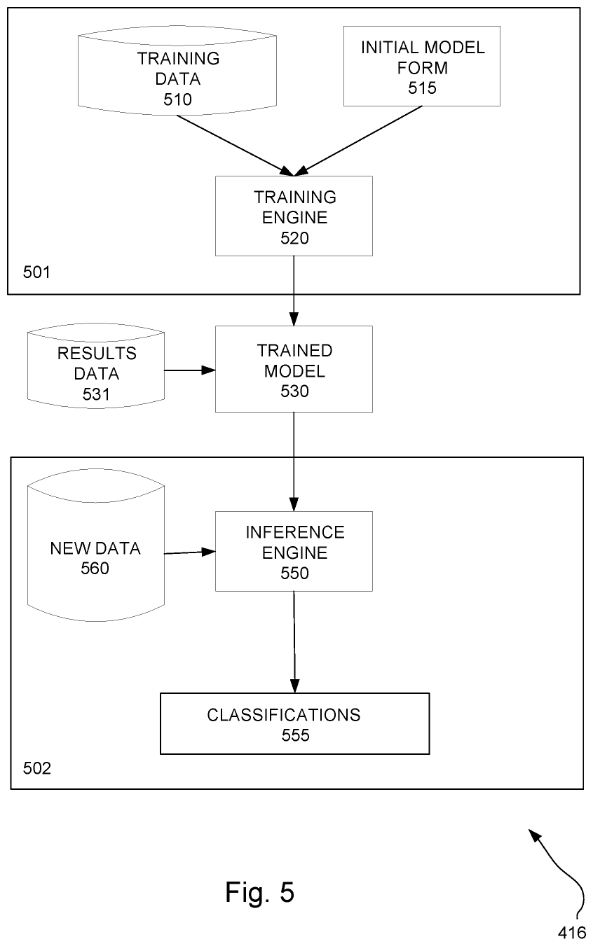

FIG. 5 is a method flow chart illustrating an exemplary computer-based method for establishing a trained prediction model and updating the trained prediction model to generate outputs according to embodiments of the subject matter disclosed herein. In this block diagram, some modules may represent functional activities, such as data collection and training, but this diagram is, nevertheless, presented in a block diagram format to convey the functional aspects of the overall analysis and prediction computing block 416 of FIG. 4. Thus, in FIG. 4, a first aggregated set of functions includes the upper half 501 of the diagram where a classifier is first established and trained for use in making predictions. Once the trained model 530 is established, the lower half 502 of the block diagram of FIG. 5 focuses on generating initial predictions to be checked against expected or historical data as well as new predictions based on new data collected.

In the upper half 501, training data 510 may be drawn from an established database of known and established sequencing data with an initial model form 515. The training data is then fed to a training engine 520 to begin establishing the trained model to be used for predictions and recommendations for health care decisions. The training data may include single cell RNA-seq data for gene variants, such as can be obtained using Perturb-seq. The model form 515 may simply be an initial “best guess” by administrators of the analysis system. As the initial training data 510 may also include outcomes such as pathogenicity predictions, a training engine 520 may begin to “train” the model form 515 by identifying specific data correlations and data trends that affect the effectiveness and accuracy of classifications from the training data 515. As all relevant and/or influential correlations are determined by the training engine 520, a trained model 530 is established.

With the trained model 530 established, an inference engine 550 may then utilize the trained model 530 along with newly collected single cell RNA-seq data. That is, a clinician or researcher may wish to use the system 400 to enhance, verify, or otherwise predict classifications for variants (e.g., pathogenic vs. benign, or pathogenic vs. likely pathogenic vs. likely benign vs. benign) based on collected data. The system may present new data 560 in the form of single cell RNA-seq data for different variants. The new data 560 may be used by the inference engine 550 that employs the trained model 530 to generate one or more classifications 555.

The inference engine 550 may be used to generate predictions based on new data that is entered as well as based on a trained model 530 established previously from training data 510. Each of the classifications discussed herein may be influenced by one or more components as discussed. Further, algorithms may be developed for one or more predicted classifications based on weightings given to each of the influential inputs. In general, any set of components may have weightings that influence any predicted outcome.

FIG. 6 is a method flow chart illustrating an exemplary computer-based method for utilizing a trained prediction model and delivering classifications based upon a trained model according to embodiments of the subject matter disclosed herein. FIG. 6 illustrates one or more algorithms that may be realized during the establishment of the trained model 530 whereby the computing system 410 may establish specific pathogenicity classifications (“outputs”) Z₁-Zₙ based on new sequencing data through its inference engine 550. That is, given inputs X₁-Xₙ, each with corresponding weighting factors Y₁-Yₙ, the inference engine 550 may utilize the trained model to generate predicted outputs Z₁-Zₙ. Generally speaking, the weighting factors may be a result of the prediction process whereby different factors are determined to be more or less influential over the prediction processes. For example, initial weighting factors may be zero as there does not exist any predictive data yet, but as predictions emerge and comparisons to reality are determined, weightings of influential factors may also emerge. These concepts may be better understood with respect to the non-limiting examples disclosed herein.

The training engine 520 may include a model trainer that includes hardware, software, or combinations of hardware and software that train one or more of the machine-learning models 530. The model trainer may implement any type of machine-learning technique to train and/or update the machine-learning models, including supervised learning, semi-supervised learning, self-supervised learning, or unsupervised learning techniques. In a non-limiting example of supervised learning, the model trainer may update trainable parameters (e.g., weights, biases, etc.) of a machine-learning model based on labeled training data. In supervised learning, both the input training data (e.g., single cell RNA-seq data for different variants). The training data includes a corresponding label, which is indicative of the desired output of the model given a particular item of input data. In a non-limiting example, the output label may be a classification regarding pathogenicity. In supervised learning, input data of the training data is provided to the machine-learning model being trained, and the machine-learning model is executed by the model trainer 155 to produce an output.

The output of the machine-learning model is then compared to the corresponding label for the input data to determine a loss, and the trainable parameters of the machine-learning model are adjusted based on the difference. The adjustment is facilitated by algorithms that guide how the parameters of the machine-learning model are changed based on the error between its predictions produced by the machine-learning model and the labels associated with the input data. One non-limiting example algorithm used to optimize the trainable parameters of the machine-learning models is gradient descent, which involves iteratively adjusting the trainable parameters by modifying them in the direction that reduces the error. The adjustments are determined by the gradient of the loss function, which measures the difference between the output predictions of the machine-learning model and the labels corresponding to the input training data. By repeatedly adjusting the parameters in the direction of steepest descent, the loss function is minimized. Various optimization algorithms may be utilized in addition to or in the alternative to gradient descent, including but not limited to stochastic gradient descent, mini-batch gradient descent, and adaptive versions such as Adam and RMSprop.

In some implementations, the trainable parameters of one or more of the machine-learning models may be updated according to supervised learning, or combinations of supervised learning and other machine-learning techniques described herein. Some non-limiting example tasks for which the machine-learning models may be trained using supervised learning include classification, regression, segmentation, and/or regression. In a non-limiting example, one or more machine-learning models described herein may include models trained to generate output pathogenicity scores that are proportional to, and therefore indicative of, likelihood of pathogenicity.

The machine-learning models may include trained or untrained models that can receive RNA-seq data as input. Such models may include, in a non-limiting example, deep convolutional neural network (CNN) models. Deep CNNs may include several layers of varying types, including but not limited to input layer(s), convolutional layers, activation functions, pooling layers, and fully connected layers, among others. Input layers include data structures that receive input data, and data to the next layer in the machine-learning model.

Convolutional layers may include trainable parameters that are used to perform convolution operations on the data provided from the previous layer in a machine-learning model. The trainable parameters may include one or more sets of learnable filters or kernels. These filters may be trained to extract features like from the input data. The depth of the output (number of feature maps) depends on the number of filters used, which may be defined as a hyperparameter of the model prior to training. Activation functions may be applied after convolution operations to introduce non-linearity into the machine-learning model. One non-limiting example of an activation function includes a Rectified Linear Unit (ReLU) activation function. Pooling layers include layers that are used to reduce the spatial dimensions of the data produced by the previous layer, and reduce the number of subsequent computations to execute the machine-learning model while providing a form of translational invariance. Non-limiting examples of pooling layers include max pooling and average pooling.

Fully connected layers of the machine-learning model(s) include one or more layers of neurons that connect to every neuron in the previous layer. In a non-limiting example, fully layers may be positioned near the output layer(s) of the machine-learning models and may be trained to produce outputs such as classification outputs and/or regression outputs. The output layer(s) of the machine-learning model(s) may be the final layers of the machine-learning model(s) that produce the final outputs of the model (e.g., classifications). In a non-limiting example for classification, various output operations such as soft-max operations may be performed to produce the final output.

According to examples of the disclosure, machine learning algorithms can be used to receive as inputs single-cell RNA-seq data for each variant, and output classifications relating to pathogenicity of each variant. Some of the machine learning algorithms that can be used in accordance with the examples of the disclosure can be convolutional neural networks (CNNs) (e.g., which can be effective in part because genetic data can be spatially correlated), support vector machines (SVMs), random forest, etc. Because nucleic acid sequences, and thus genes and pseudogenes of interest, can have a sequential character, recurrent neural networks (RNNs) can be conducive for use in such applications, because RNNs make use of sequential information in their operation in that the output of a RNN for a given element in a sequence depends on the operations of the RNN during the previous one or more elements in the sequence. Such operation that is grounded in sequential operations aligns with the sequential character of nucleic acids.

In an example process, a machine learning model (e.g., RNN) can be used to determine classifications according to examples of the disclosure. Input data (e.g., an input vector) can correspond to single cell RNA-seq data, as described above. For example, input vector can be inputted to the model that has been trained with RNA-seq data obtained through, for example, a Perturb-seq process described herein, and corresponding classifications and/or confidence scores can be output by the model. The RNA-seq data, and classifications, used to train the model can range in sample size from 100 to 100,000 (e.g., RNA-seq data for 1,000, 5,000, 10,000, 20,000, 30,000 or more single cells), each corresponding to a variant of a gene. Training data can include data from samples with pathogenic and non-pathogenic variants. In some examples, the model can output, along with the classification, indications of the confidence with which those classifications are made (e.g., confidence scores ranging from 0 (least confident) to 1.0 (most confident), or activation values between 0 and 1 such that an activation value between 0 and 0.5 indicates that the sample is regarded as negative for pathogenicity (0 being most-confidently negative, and 0.5 being least-confidently negative), and an activation value between 0.5 and 1 indicates that the sample is regarded as positive for pathogenicity (1 being most-confidently positive, and 0.51 being least-confidently positive)). In such examples, the model can be trained with confidence scores, in addition to the single cell RNA-seq data, so as to be able to produce confidence scores as outputs when used in the process. Such confidence scores may inform classifications, such that a classification is accepted or deemed usable if the confidence score exceeds a threshold. Pathogenic classifications may be assigned based in part on confidence scores, or provided with a confidence score. In an alternative process flow, models only output classifications if those classifications are associated with relatively high confidence levels (e.g., confidence levels greater than a threshold confidence level, such as 0.8 or 0.9) according to examples of the disclosure. For example, if a model is able to produce classifications at such a high confidence level, then it outputs those classifications (which can be, for example, inserted into a report, medical record, etc.). However, if the model is not able to produce classifications with acceptable confidence levels (e.g., the confidence level is less than or equal to the above threshold confidence level), then the model does not provide classifications, and instead can be, for example, flagged for review.

In some embodiments, the input of the machine learning model comprises, or alternatively consists essentially of, or yet further consists of one or more of a transcriptomic, genomic, genetic, proteomic, and/or epigenetic data from a plurality of single cells. In some embodiments, the transcriptomic data comprises, or alternatively consists essentially of, or yet further consists of single-cell RNA-seq data. In some embodiments, the genomic data comprises, or alternatively consists essentially of, or yet further consists of single-cell whole genome sequencing data. In some embodiments, the proteomic data comprises, or alternatively consists essentially of, or yet further consists of single-cell mass spectroscopy data. In some embodiments, the epigenetic data comprises, or alternatively consists essentially of, or yet further consists of single-cell bisulfite sequencing data.

### Perturb-Seq

In some embodiments, Perturb-seq is used to make and detect a plurality of variants in a gene as described in U.S. Pat. No. 11,214,797, which is incorporated herein in its entirety. Perturb-seq combines single cell RNA-seq and CRISPR/Cas9 based perturbations identified by unique polyadenylated barcodes to perform many, in certain embodiments tens of thousands, of such assays in a single pooled experiment. In some embodiments, Perturb-seq is used to introduce mutations in a gene of interest. In some embodiments, mutations are introduced at every position of a gene of interest using Perturb-seq, wherein each individual cell has only one mutation. In some embodiments, Perturb-seq can be used to test transcriptional phenotypes caused by genetic interactions by randomly integrating more than one sgRNA in each cell.

By combining droplet based single-cell sequencing with CRISPR-Cas based perturbations, the Perturb-seq approach allows researchers to perform thousands of assays in a single pooled experiment. Leveraging the discrete nature of the single cell measurements, the screening approach also can resolve novel phenotypes such as the effect of a perturbation on cell type composition or cell cycle phase, and filter unperturbed cells whose presence otherwise dilutes the measured effect in population measurements. In some embodiments, Perturb-seq can test combinatorial effects of gene variants by randomly integrating more than one sgRNA in each cell.

In one aspect, the present disclosure provides for a method of mapping effects of gene variants, comprising, or alternatively consisting essentially of, or yet further consisting of introducing at least 1, 2, 3, 4 or more single-order or combinatorial perturbations to a plurality of cells in a population of cells, wherein each cell in the plurality of the cells receives at least 1 perturbation; measuring comprising, or alternatively consisting essentially of, or yet further consisting of: detecting genomic, genetic, proteomic, epigenetic and/or phenotypic differences in single cells compared to one or more cells that did not receive any perturbation, and detecting the perturbation(s) in single cells; and determining measured differences relevant to the perturbations by applying a machine learning model accounting for co-variates to the measured differences, whereby effects of individual variants/combination of variants are determined.

In some embodiments, the measuring in single cells comprises, or alternatively consists essentially of, or yet further consists of single cell sequencing. In some embodiments, single cell sequencing comprises, or alternatively consists essentially of, or yet further consists of using cell barcodes, whereby the cell-of-origin of each RNA is recorded. In some embodiments, single cell sequencing comprises, or alternatively consists essentially of, or yet further consists of unique molecular identifiers (UMI), whereby the capture rate of the measured signals, such as transcript copy number or probe binding events, in a single cell is determined. In some embodiments, the machine learning model of the disclosure comprises, or alternatively consists essentially of, or yet further consists of accounting for the capture rate of measured signals, whether the perturbation actually affected the cell (phenotypic impact), the presence of subpopulations of either different cells or cell states, and/or analysis of matched cells without any perturbation.

In some embodiments, single-order or combinatorial perturbations comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 perturbations. The perturbation(s) may target genes in a pathway or intracellular network.

In some embodiments, the measuring may comprise detecting the transcriptome of each of the single cells. In some embodiments, the perturbation(s) may comprise one or more genetic perturbation(s). The perturbation(s) may comprise one or more epigenetic or epigenomic perturbation(s). At least one perturbation may be introduced with RNAi-or a CRISPR-Cas system. In some embodiments, at least one perturbation may be introduced via a chemical agent, biological agent, an intracellular spatial relationship between two or more cells, an increase or decrease of temperature, addition or subtraction of energy, electromagnetic energy, or ultrasound.

In some embodiments, the cell comprises, or alternatively consists essentially of, or yet further consists of a cell in a model non-human organism, a model non-human mammal that expresses a Cas protein, a mouse that expresses a Cas protein, a mouse that expresses Cpf1 (Cas12a), a cell in vivo or a cell ex vivo or a cell in vitro. In some embodiments, the cell comprises, or alternatively consists essentially of, or yet further consists of a human cell.

In some embodiments, the perturbing or perturbation comprises, or alternatively consists essentially of, or yet further consists of genetic perturbing. In some embodiments, the perturbing or perturbation comprises, or alternatively consists essentially of, or yet further consists of single-order perturbations. In some embodiments, the perturbing or perturbation comprises, or alternatively consists essentially of, or yet further consists of combinatorial perturbations. In some embodiments, the perturbing or perturbation comprises, or alternatively consists essentially of, or yet further consists of gene knock-down, gene knock-out, gene activation, gene insertion, or regulatory element deletion. In some embodiments, the perturbing or perturbation comprises, or alternatively consists essentially of, or yet further consists of genome-wide perturbation.

In some embodiments, the perturbing or perturbation comprises, or alternatively consists essentially of, or yet further consists of performing CRISPR-Cas-based perturbation. In some embodiments, the perturbing or perturbation comprises, or alternatively consists essentially of, or yet further consists of performing pooled single or combinatorial CRISPR-Cas-based perturbation with a genome-wide library of sgRNAs. In some embodiments, the perturbations are of a selected group of targets based on similar pathways or network of targets.

In some embodiments, the perturbing or perturbation comprises, or alternatively consists essentially of, or yet further consists of performing pooled combinatorial CRISPR-Cas-based perturbation with a genome-wide library of sgRNAs. Each sgRNA may be associated with a unique perturbation barcode. Each sgRNA may be co-delivered with a reporter mRNA comprising, or alternatively consisting essentially of, or yet further consisting of the unique perturbation barcode (or sgRNA perturbation barcode).

In some embodiments, the perturbing or perturbation comprises, or alternatively consists essentially of, or yet further consists of subjecting the cell to an increase or decrease in temperature. In some embodiments, the perturbing or perturbation comprises, or alternatively consists essentially of, or yet further consists of subjecting the cell to a chemical agent. In some embodiments, the perturbing or perturbation comprises, or alternatively consists essentially of, or yet further consists of subjecting the cell to a biological agent. The biological agent may be a toll like receptor agonist or cytokine. In some embodiments, the perturbing or perturbation comprises, or alternatively consists essentially of, or yet further consists of subjecting the cell to a chemical agent, biological agent and/or temperature increase or decrease across a gradient.

In some embodiments, the cell is in a microfluidic system. In some embodiments, the cell is in a droplet. In some embodiments, the population of cells is sequenced by using microfluidics to partition each individual cell into a droplet containing a unique barcode, thus allowing a cell barcode to be introduced.

In some embodiments, the perturbing or perturbation comprises, or alternatively consists essentially of, or yet further consists of transforming or transducing the cell or a population that includes and from which the cell is isolated with one or more genomic sequence-perturbation constructs (“mutant expression vectors”) that perturbs a genomic sequence in the cell. The sequence-perturbation construct may be a viral vector, e.g., a lentivirus vector. In some embodiments, the perturbing or perturbation comprises, or alternatively consists essentially of, or yet further consists of multiplex transformation or transduction with a plurality of genomic sequence-perturbation constructs.

In another aspect, or in alternative embodiments of aspects described herein, the present disclosure provides for a method wherein proteins or transcripts expressed in single cells are determined in response to a perturbation, wherein the proteins or transcripts are detected in the single cells by binding of more than one labeling ligand comprising, or alternatively consisting essentially of, or yet further consisting of an oligonucleotide tag, and wherein the oligonucleotide tag comprises, or alternatively consists essentially of, or yet further consists of a unique constituent identifier (UCI) specific for a target protein or transcript. In some embodiments, single cells are fixed in discrete particles. In some embodiments, discrete particles may be washed and sorted, such that cell barcodes are added, e.g., sgRNA perturbation barcode. In some embodiments, the oligonucleotide tag and sgRNA perturbation barcode may comprise a universal ligation handle sequence, whereby a unique cell barcode may be generated by split-pool ligation. In some embodiments, the labeling ligand may comprise an oligonucleotide label comprising, or alternatively consisting essentially of, or yet further consisting of a regulatory sequence configured for amplification by T7 polymerase. In some embodiments, the labeling ligands may comprise oligonucleotide sequences configured to hybridize to a transcript specific region. In some embodiments, proteins and/or RNAs are detected after perturbation. In some embodiments, the oligonucleotide label further comprises, or alternatively consists essentially of, or yet further consists of a photocleavable linker. In some embodiments, the oligonucleotide label further comprises, or alternatively consists essentially of, or yet further consists of a restriction enzyme site between the labeling ligand and unique constituent identifier (UCI). In some embodiments, the ligation handle comprises, or alternatively consists essentially of, or yet further consists of a restriction site for producing an overhang complementary with a first index sequence overhang, and wherein the method further comprises, or alternatively consists essentially of, or yet further consists of digestion with a restriction enzyme. In some embodiments, the ligation handle comprises, or alternatively consists essentially of, or yet further consists of a nucleotide sequence complementary with a ligation primer sequence and wherein the overhang complementary with a first index sequence overhang is produced by hybridization of the ligation primer to the ligation handle. In some embodiments, the method further comprises, or alternatively consists essentially of, or yet further consists of quantitating the relative amount of UCI sequence associated with a first cell to the amount of the same UCI sequence associated with a second cell, whereby the relative differences of a cellular constituent between cells are determined. In some embodiments, the labeling ligand comprises, or alternatively consists essentially of, or yet further consists of an antibody or an antibody fragment. In some embodiments, the antibody fragment may be a nanobody, Fab, Fab′, (Fab′)₂, Fv, ScFv, diabody, triabody, tetrabody, Bis-scFv, minibody, Fab₂, or Fab₃ fragment. The labeling ligand may comprise an aptamer. The labeling ligand may be a nucleotide sequence complementary to a target sequence.

In some embodiments, single-cell sequencing comprises, or alternatively consists essentially of, or yet further consists of whole transcriptome amplification.

The method in aspects of the disclosure may comprise comparing an RNA profile of the perturbed cell with any mutations in the cell to also correlate phenotypic or transcriptome profile and genotypic profile.

The method in aspects of this disclosure may comprise performing RNAi-or CRISPR-Cas-based perturbation. The method may comprise an array-format or pool-format perturbation. The method may comprise pooled single or combinatorial CRISPR-Cas-based perturbation with a genome-wide library of sgRNAs. The method may comprise pooled combinatorial CRISPR-Cas-based perturbation with a genome-wide library of sgRNAs.

The perturbation of the population of cells may be performed in vivo. The perturbation of the population of cells may be performed ex vivo and the population of cells may be adoptively transferred to a subject. The population of cells may comprise tumor cells. The method may comprise a lineage barcode associated with single cells, whereby the lineage or clonality of single cells may be determined.

The perturbing may be across a library of cells to thereby obtain RNA level and/or optionally protein level, whereby cell-to-cell circuit data at genomic or transcript or expression level is determined. The library of cells may comprise or is from a tissue sample. The tissue sample may comprise or is from a biopsy from a mammalian subject. The mammalian subject may comprise a human subject. The biopsy may be from a tumor. The method may further comprise reconstructing cell-to-cell circuits.

### Methods of the Disclosure

An aspect of the disclosure is directed to a method of determining the effects of a plurality of variants in a gene comprising, or alternatively consisting essentially of, or yet further consisting of: a) generating a mutant expression vector for each of the plurality of variants; b) expressing each variant in a cell in a plurality of single cells; and c) sequencing the RNA expressed in each single cell and evaluating expression patterns that result in each single cell to generate single cell RNA sequencing data.

In some embodiments, the method further comprises, or alternatively consists essentially of, or yet further consists of evaluating the single cell RNA sequencing data to determine the pathogenicity of each variant.

In some embodiments, evaluating the single cell RNA sequencing data comprises, or alternatively consists essentially of, or yet further consists of use of machine learning.

In some embodiments, variants are classified as either pathogenic, likely pathogenic, likely benign, or benign.

In some embodiments, each cell of the plurality of single cells is expresses a single variant of the plurality of variants.

In some embodiments, each single variant of the plurality of variants is expressed in more than one cell, thereby creating redundancy of the single cell RNA sequencing data.

In some embodiments, the plurality of single cells comprises, or alternatively consists essentially of, or yet further consists of at least 30,000, at least 40,000, at least 50,000, at least 60,000, at least 70,000, at least 80,000, at least 90,000 or at least 100,000 single cells.

In some embodiments, the plurality of variants comprises, or alternatively consists essentially of, or yet further consists of at least 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or at least 3000 variants.

In some embodiments, the gene is selected from genes associated with rare disease.

In some embodiments, the gene is selected from genes associated with oncology.

In some embodiments, the mutant expression vector is generated using CRISPR.

In some embodiments, the mutant expression vector is generated using CRISPRi.

In some embodiments, the mutant expression vector is generated using RNAi.

In some embodiments, the RNA from each single cell is identified using barcoding and sequencing.

In some embodiments, the method further comprises repeating steps a)-c) in more than one set of primary cells or cell lines.

Another aspect of the disclosure is directed to a method of simultaneously determining the effects of a gene comprising, or alternatively consisting essentially of, or yet further consisting of: a) perturbing the function of a gene in a cell in a plurality of single cells; and b) sequencing the RNA expressed in each single cell and evaluating expression patterns that result in each single cell to generate single cell RNA sequencing data.

In some embodiments, perturbing the function of a gene is achieved by CRISPR, CRISPRi or RNAi.

In some embodiments, the method further comprises evaluating the single cell RNA sequencing data to determine the pathogenicity of each perturbed gene.

In some embodiments, perturbing the function of a gene comprises, or alternatively consists essentially of, or yet further consists of using one or more drug compounds. In some embodiments, a different drug compound is administered to each single cell.

In some embodiments, the plurality of drug compounds comprises, or alternatively consists essentially of, or yet further consists of at least 100, at least 200, at least 300, at least 500, at least 1,000, at least 2,000, at least 3,000 at least 4,000, at least 5,000, or at least 10,000 compounds.

In some embodiments, evaluating the single cell RNA sequencing data comprises, or alternatively consists essentially of, or yet further consists of use of machine learning.

Another aspect of the disclosure is directed to a method for determining the effects of a plurality of variants in a gene comprising, or alternatively consisting essentially of, or yet further consisting of: a) expressing a plurality of mutant expression vectors in a plurality of single cells, wherein each mutant expression vector in the plurality of mutant expression vectors is capable of creating a different variant in the gene; b) sequencing the RNA expressed in each single cell to generate single cell RNA sequencing data; and c) determining the pathogenicity of each variant using machine learning.

A number of embodiments have been described. Nevertheless, it will be understood that various modifications may be made. Accordingly, other embodiments are within the scope of the following claims.

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present technology has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A method of determining the effects of a plurality of variants in a gene comprising:
a) generating a mutant expression vector for each of the plurality of variants;
b) expressing each variant in a cell in a plurality of single cells; and
c) sequencing the RNA expressed in each single cell and evaluating expression patterns that result in each single cell to generate single cell RNA sequencing data.

2. The method of claim 1, further comprising evaluating the single cell RNA sequencing data to determine the pathogenicity of each variant.

3. The method of claim 1, wherein evaluating the single cell RNA sequencing data comprises use of machine learning.

4. The method of claim 1, variants are classified as either pathogenic, likely pathogenic, likely benign, or benign.

5. The method of claim 1, wherein each cell of the plurality of single cells is configured to express a single variant of the plurality of variants.

6. The method of claim 1, wherein each single variant of the plurality of variants is expressed in more than one cell, thereby creating redundancy of the single cell RNA sequencing data.

7. The method of claim 1, wherein the plurality of single cells comprises at least 30,000, at least 40,000, at least 50,000, at least 60,000, at least 70,000, at least 80,000, at least 90,000 or at least 100,000 single cells.

8. The method of claim 1, wherein the plurality of variants comprises at least 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or at least 3000 variants.

9. The method of claim 1, wherein the gene is selected from genes associated with rare disease or genes associated with oncology.

10. (canceled)

11. The method of claim 1, wherein the mutant expression vector is generated using CRISPR, CRISPRi or RNAi.

12. (canceled)

13. (canceled)

14. The method of claim 1, wherein the RNA from each single cell is identified using barcoding and sequencing.

15. The method of claim 1, further comprising repeating steps a)-c) in more than one set of primary cells or cell lines.

16. A method of determining the effects of a gene comprising:
a) perturbing the function of a gene in a cell in a plurality of single cells; and
b) sequencing the RNA expressed in each single cell and evaluating expression patterns that result in each single cell to generate single cell RNA sequencing data.

17. The method of claim 16, further comprising evaluating the single cell RNA sequencing data to determine the pathogenicity of each perturbed gene.

18. The method of claim 17, wherein perturbing the function of a gene comprises using one or more drug compounds.

19. The method of claim 18, wherein a different drug compound is administered to each single cell.

20. The method of claim 18, wherein the plurality of drug compounds comprises at least 100, at least 200, at least 300, at least 500, at least 1,000, at least 2,000, at least 3,000 at least 4,000, at least 5,000, or at least 10,000 compounds.

21. The method of claim 16, wherein evaluating the single cell RNA sequencing data comprises use of machine learning.

22. A method comprising:
a) receiving RNA-seq data for a plurality of cells, each cell expressing a variant generated using a mutant expression vector; and
b) using the RNA-seq data to train a machine learning model to receive, as input, RNA-seq data and provide, as output, a pathogenicity classification.

23. A method comprising:
a) receiving RNA-seq data for a cell; and
b) providing the RNA-seq data to a machine learning model to generate a pathogenicity classification for a variant, the machine learning model trained according to the method of claim 22.
